(19) United States
(12) Patent Application Publication
(10) Pub. No.: US 2007/0250001 A1
(43) Pub. Date: Oct. 25, 2007

(54) Guidewire Separator Device and Method of Use

(76) Inventors: Pierre Hilaire, Marly Le Roi (FR); Machiel Van Der Leest, Paris (FR); Thierry Royer, Paris (FR)

Correspondence Address: GREGORY SMITH & ASSOCIATES, 3900 NEWPARK MALL ROAD, 3RD FLOOR, NEWARK, CA, 94560, US

(21) Appl. No.: 11/597,339

(22) PCT Filed: May 21, 2005
(86) PCT No.: PCT/US05/18148

§ 371 Date: Nov. 21, 2006

Related U.S. Application Data
(60) Provisional application No. 60/573,686, filed on May 21, 2004.

(51) Int. Cl. A61M 31/00

(52) U.S. Cl. 604/103.04

(57) ABSTRACT

A guidewire separator device and methods are described for resolving guidewire entanglement and for introducing guidewires into branch vessels when performing angioplasty or stenting of a bifurcated artery. The device has an elongate tubular distal portion joined to an elongate proximal portion. A first lumen extends through the tubular distal portion of the device, with a first distal guidewire port located near a distal end of the distal portion and a first proximal guidewire port located near a proximal end of the distal portion. A second lumen extends through the tubular distal portion of the device, with a second distal guidewire port located near a distal end of the distal portion and a second proximal guidewire port located near a proximal end of the second lumen. One or both of the lumens may continue through the proximal portion of the device. Optionally, the device includes one or more longitudinal slits to externalize the guidewires from the lumens during withdrawal of device.

### FIELD OF THE INVENTION

The present invention relates generally to guidewires, catheters and catheter systems, such as those used for performing angioplasty and vascular stenting. More particularly it relates to a guidewire separator device and method of use for catheterizing or stenting a vessel at a bifurcation or sidebranch of the vessel.

### BACKGROUND OF THE INVENTION

One of the techniques commonly used for performing angioplasty and vascular stenting of a vessel at a bifurcation or sidebranch of the vessel is known as the “kissing balloons” technique. Improvements to the “kissing balloons” technique are disclosed in commonly owned and copending U.S. patent application for Catheter System for Stenting Bifurcated Vessels, Ser. No. 10/833494, filed on Apr. 27, 2004. This application and all patents and patent applications referred to herein are incorporated by reference in their entirety.

One common method of performing the “kissing balloons” technique involves inserting a first guidewire into the first branch of the bifurcated vessel, then inserting a second guidewire into the second branch of the bifurcated vessel. A stent deployment catheter or catheter system with one or more stents mounted on it is then introduced over the first and second guidewires to deploy the stent or stents at the bifurcation. Difficulties can arise when attempting to position the first and second guidewires in the branches of the bifurcated vessel. Because of the manipulations that may be necessary to maneuver the guidewires into each of the branches, there is a possibility that the guidewires may twist around one another and become entangled. This problem is more common when one or both of the branches is difficult to engage with the guidewire, requiring additional manipulations to maneuver the guidewires into their respective branches of the bifurcation. When this occurs, the guidewires must be disentangled and separated from one another before the stent deployment catheter can be inserted.

U.S. Pat. No. 6,682,536 describes a guidewire introducer sheath that is intended for introducing the first and second guidewires in a method that avoids the problem of the guidewires becoming entangled. This method must be used preemptively to avoid guidewire entanglement and cannot readily be used to separate the guidewires after they have become entangled. Routine use of this method would add an unnecessary device and unnecessary method steps to many procedures where it may not have been needed. In addition, the configuration in some embodiments of the guidewire introducer sheath requires that exchange length guidewires or guidewire extensions be used for the procedure, which also adds more expense and complexity to the procedure.

The following patents and patent applications also relate to catheters and catheter systems for performing angioplasty and stenting of bifurcated vessels.

U.S. Pat. No. 6,579,312 Stent and catheter assembly and method for treating bifurcations

U.S. Pat. No. 6,540,779 Bifurcated stent with improved side branch aperture and method of making same

U.S. Pat. No. 6,520,988 Endolumenal prosthesis and method of use in bifurcation regions of body lumens

U.S. Pat. No. 6,508,836 Stent and catheter assembly and method for treating bifurcations

U.S. Pat. No. 6,494,875 Bifurcated catheter assembly

U.S. Pat. No. 6,475,208 Bifurcated catheter assembly

U.S. Pat. No. 6,428,567 Stent and catheter assembly and method for treating bifurcations

U.S. Pat. No. 6,387,120 Stent and catheter assembly and method for treating bifurcations

U.S. Pat. No. 6,383,213 Stent and catheter assembly and method for treating bifurcations

U.S. Pat. No. 6,371,978 Bifurcated stent delivery system having retractable sheath

U.S. Pat. No. 6,361,544 Stent and catheter assembly and method for treating bifurcations

U.S. Pat. No. 6,325,826 Extendible stent apparatus

U.S. Pat. No. 6,264,682 Bifurcated stent delivery system having retractable sheath

U.S. Pat. No. 6,258,073 Bifurcated catheter assembly

U.S. Pat. No. 6,254,593 Bifurcated stent delivery system having retractable sheath

U.S. Pat. No. 6,221,098 Stent and catheter assembly and method for treating bifurcations

U.S. Pat. No. 6,210,380 Bifurcated catheter assembly

U.S. Pat. No. 6,165,195 Stent and catheter assembly and method for treating bifurcations

U.S. Pat. No. 6,142,973 Y-shaped catheter

U.S. Pat. No. 6,117,117 Bifurcated catheter assembly

U.S. Pat. No. 6,086,611 Bifurcated stent

U.S. Pat. No. 5,720,735 Bifurcated endovascular catheter

U.S. Pat. No. 5,669,924 Y-shuttle stent assembly for bifurcating vessels and method of using the same

U.S. Pat. No. 5,613,980 Bifurcated catheter system and method

U.S. Pat. No. 6,013,054 Multifurcated balloon catheter

U.S. Pat. No. 4,896,670 Kissing balloon catheter

U.S. Pat. No. 5,395,352 Y-adaptor manifold with pinch valve for an intravascular catheter

U.S. Pat. No. 6,129,738 Method and apparatus for treating stenoses at bifurcated regions

U.S. Pat. No. 6,544,219 Catheter for placement of therapeutic devices at the ostium of a bifurcation of a body lumen

U.S. Pat. No. 6,494,905 Balloon catheter

U.S. Pat. No. 5,749,825 Means method for treatment of stenosed arterial bifurcations

U.S. Pat. No. 5,320,605 Multi-wire multi-balloon catheter

U.S. Pat. No. 6,099,497 Dilatation and stent delivery system for bifurcation lesions

U.S. Pat. No. 5,720,735 Bifurcated endovascular catheter

U.S. Pat. No. 5,906,640 Bifurcated stent and method for the manufacture and delivery of same

U.S. Pat. No. 5,893,887 Stent for positioning at junction of bifurcated blood vessel and method of making

U.S. Pat. No. 5,755,771 Expandable stent and method of delivery of same

US 20030097169A1 Bifurcated stent and delivery system

US 20030028233A1 Catheter with attached flexible side sheath

US 20020183763A1 Stent and catheter assembly and method for treating bifurcations

US 20020156516A1 Method for employing an extendible stent apparatus

US 20020116047A1 Extendible stent apparatus and method for deploying the same

US 20020055732A1 Catheter assembly and method for positioning the same at a bifurcated vessel

WO 9944539A2 Dilatation and stent delivery system for bifurcation lesions

WO 03053507 Branched balloon catheter assembly

WO 9924104 Balloon catheter for repairing bifurcated vessels

WO 0027307 The sheet expandable trousers stent and device for its implantation

FR 2733689 Endoprosthesis with installation device for treatment of blood-vessel bifurcation stenosis

### SUMMARY OF THE INVENTION

In keeping with the foregoing discussion, it would be desirable therefore to provide a device and methods that can be used electively to either prevent guidewire entanglement or, if the need arises, to quickly and easily separate the entangled guidewires and/or to assist in proper placement of the guidewires into their respective branches of the bifurcation. Preferably, the device and methods would not require the use of exchange length guidewires or guidewire extensions. Such a device and methods would not disrupt the normal routine in the majority of cases where the problem of guidewire entanglement does not occur, but could be readily available for use in resolving problems when they arise.

A guidewire separator device and methods of use are described for preventing guidewire entanglement and for quickly and easily separating entangled guidewires and assisting in proper placement of the guidewires into their respective branch vessels when performing angioplasty or stenting of a bifurcated artery. The guidewire separator device has an elongate tubular distal portion joined to an elongate proximal portion. A first lumen extends through at least the tubular distal portion of the device, with a first distal guidewire port located near a distal end of the distal portion and a first proximal guidewire port located near a proximal end of the distal portion. A second lumen extends through at least the tubular distal portion of the guidewire separator device, with a second distal guidewire port located near a distal end of the distal portion and a second proximal guidewire port located near a proximal end of the second lumen. Optionally, the guidewire separator device includes a longitudinal slit in a sidewall of the device that communicates with the second lumen.

A method for separating two guidewires that have become entangled using the guidewire separator device includes inserting a proximal end of the first guidewire into the first lumen of the device; inserting a proximal end of the second guidewire into the second lumen of the device; advancing the device distally along the first guidewire and the second guidewire to separate and disentangle the guidewires; withdrawing the device proximally along the guidewires and externalizing the second guidewire from the second lumen through a longitudinal slit in a sidewall of the device. A method for introducing two guidewires into a bifurcated vessel using the guidewire separator device includes preloading the second guidewire into the second lumen of the device; inserting the first guidewire into a first branch of the bifurcated vessel; inserting a proximal end of the first guidewire into the first lumen of the device; advancing the device into the bifurcated vessel along the first guidewire; advancing the second guidewire though the second lumen and into a second branch of the bifurcated vessel; and withdrawing the device proximally along the guidewires and externalizing the second guidewire from the second lumen through a longitudinal slit in a sidewall of the device.

In addition to the “kissing balloons” technique of angioplasty and stenting, the guidewire separator device and methods of the present invention will find application in other areas where two guidewires, catheters or other elongated devices are used side-by-side within the patient's vasculature or other organ systems of the body.

### BRIEF DESCRIPTION OF THE DRAWINGS

FIG. 1 shows a guidewire separator device according to the present invention.

FIG. 2 shows the insertion and withdrawal of the guidewire separator device over two guidewires that have been previously inserted into the patient.

FIG. 3 shows the entangled distal ends of the guidewires within the bifurcated vessel.

FIG. 4 shows the guidewire separator device within the bifurcated vessel after separating the entangled distal ends of the guidewires.

FIG. 5 shows an alternate embodiment of the guidewire separator device.

FIG. 6 shows an alternate embodiment of the guidewire separator device.

FIG. 7 shows an embodiment of the guidewire separator device configured to function as a guidewire introducer.

FIG. 8 shows a cross section of the guidewire separator device of FIG. 7 taken at section line 8-8.

FIGS. 9-15 illustrate a method of using the guidewire separator device of FIG. 7.

FIG. 16 shows another embodiment of the guidewire separator device configured to function as a guidewire introducer.

FIG. 17 shows a cross section of the guidewire separator device of FIG. 16 taken at section line 17-17.

FIG. 18 shows a cross section of the guidewire separator device of FIG. 16 taken at section line 18-18.

FIG. 19 shows a cross section of the guidewire separator device of FIG. 16 taken at section line 19-19.

### DESCRIPTION OF THE INVENTION

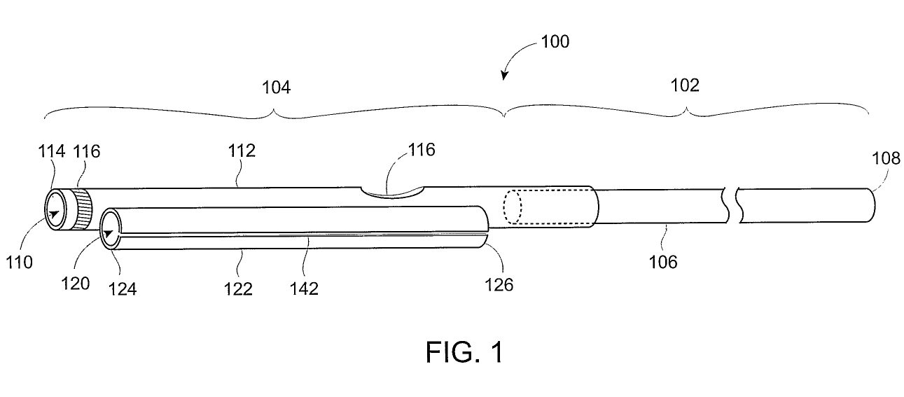

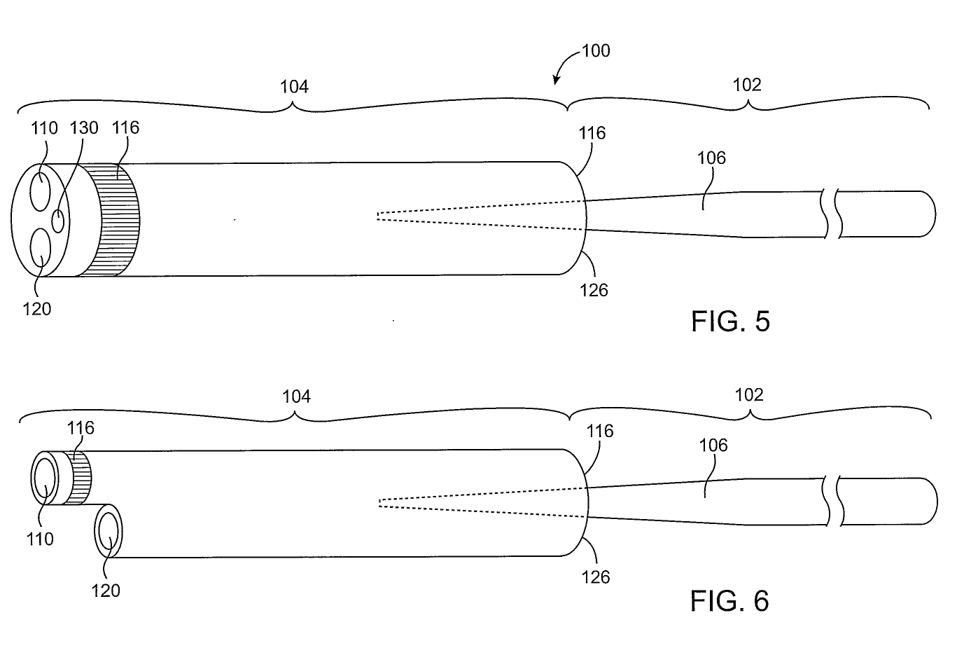

FIG. 1 shows a guidewire separator device 100 according to the present invention. The guidewire separator device 100 is constructed with a distal portion 104 having a first lumen 110 and a second lumen 120. The distal portion 104 is mounted to or otherwise connected to a proximal portion 102. The distal portion 104 may be formed of two tubes 112, 122 joined together side-by-side or it may be formed from a two-lumen tube. In a preferred embodiment, the distal portion 104 is constructed of a flexible polymer extrusion, preferably with a polymer having a relatively low coefficient of friction. Suitable materials include, but are not limited to polyethylene, polypropylene, polyolefins, polyamides, polyamide copolymers, PTFE, FEP, and any extrudable medical grade polymer with a suitable combination of strength, flexibility and friction characteristics. The proximal portion 102 may be configured as a straight wire or stylet 106 joined to the distal portion 104, which may be formed from stainless steel, a cobalt alloy, or a superelastic nickel-titanium or titanium-molybdenum alloy, or the like. Alternatively, the proximal portion 102 may be constructed of a rigid polymer or a reinforced polymer composite. One convenient way of constructing the guidewire separator device 100 is to insert the distal end of the stylet 106 that forms the proximal portion 102 into the first lumen 110 of the distal portion 104 and bonding the proximal and distal portions 102, 104 together with an adhesive or the like. Other constructions are also possible. For example, the distal portion 104 may be formed of a three-lumen polymer extrusion, the third lumen 130 being sized to fit the distal end of the proximal portion 102 for bonding. Optionally, the distal end of the proximal portion 102 may be taper ground to provide a smooth stiffness transition at the junction between the proximal portion 102 and the distal portion 104. FIGS. 5 and 6 show two alternate embodiments of the guidewire separator device 100 utilizing these features.

The first lumen 110 has a first guidewire entry port 114 located at or near a distal end of the distal portion 104 and a first guidewire exit port 116 near a proximal end of the distal portion 104. The first guidewire exit port 116 may be formed by skiving or cutting a hole in the sidewall of the distal portion 104 that communicates with the first lumen 110, just distal to where the proximal portion 102 terminates, as shown in FIG. 1. Optionally, an internal ramp or deflector (not shown) may be formed in the first lumen 110 to urge the proximal end of an inserted guidewire out through the first guidewire exit port 116. Alternatively, the proximal end of the first lumen 110 may simply be open at the proximal end of the distal portion 104 to create the first guidewire exit port 116, as shown in FIGS. 5 and 6. The second lumen 120 has a second guidewire entry port 124 located at or near the distal end of the distal portion 104 and a second guidewire exit port 126 near the proximal end of the distal portion 104. The second guidewire exit port 126 may be formed by leaving the proximal end of the second lumen 120 open, as shown in FIGS. 1, 5 and 6. Alternatively, the second guidewire exit port 126 may be formed by skiving or cutting a hole in the sidewall of the distal portion 104 that communicates with the second lumen 120.

Optionally, the distal portion 104 may have one or more longitudinal slits 142 that communicate with the first and/or second lumens 110, 120 to allow a guidewire to be inserted and/or removed from the lumen through the side of the distal portion 104. Alternatively, the sidewall of the distal portion 104 may be made splittable along a longitudinal line to allow a guidewire to be removed from the lumen through the side of the distal portion 104.

In a preferred embodiment, the first lumen 110 extends approximately 1-2 cm distally beyond the second guidewire entry port 124. Preferably, a radiopaque marker ring 116 or the like is attached near the distal end of the first lumen 110 or elsewhere on the distal portion 104 of the device 100. Optionally, two or more radiopaque markers with different locations and/or geometries may be attached on the distal portion 104 to help determine the location and orientation of the device 100 using fluoroscopy. Preferably, the distal portion 104 of the device 100 is unencumbered by other structure such as balloons or stents that would interfere with its function as a guidewire separator and introducer.

The dimensions of the guidewire separator device 100 are variable depending on the clinical application and the anatomy where it is intended to be used. By way of example, a guidewire separator device 100 for use in angioplasty or stenting of coronary arteries will preferably have a distal portion 104 with a length of approximately 15-20 cm and a proximal portion 102 with a length of approximately 100-160 cm. The internal diameter of the first and second lumens 110, 120 will be large enough and have sufficient clearance for a smooth sliding fit over the guidewires the device 100 is intended to be used with. Common sizes for coronary guidewires are currently 0.010, 0.014 and 0.018 inches (approximately 0.25, 0.36 and 0.46 mm) nominal diameter, however the device 100 can easily be modified to work with other diameters of guidewires, such as those used for carotid or peripheral vascular stenting. The internal diameters of the first and second lumens 110, 120 may be different if two guidewires of different diameters are to be use. The outer diameter of the distal portion 104 will preferably be as small as practically possible given that it must accommodate the first and second lumens 110, 120 and have sufficient wall thickness for structural strength and kink resistance. For use with two 0.014 inch (0.36 mm) diameter guidewires, the distal portion 104 can be constructed with an outer diameter of approximately 1.0 mm. At a maximum, the outer diameter of the distal portion 104 must be small enough to fit through the lumen of the guiding catheter that it is intended to be used with. For example, 6 French guiding catheters (approximately 2 mm outer diameter) typically have an internal diameter of approximately 1.68-1.80 mm. The proximal portion 102 will preferably have a diameter of approximately 0.25-0.50 mm.

In an alternate configuration, the proximal portion 102 of the guidewire separator device 100 may be configured as a tube with a lumen aligned with the first lumen 110 of the distal portion 104. The tubular proximal portion 102 will have an outer diameter of approximately 0.5-1.0 mm with an internal lumen of sufficient size for passage of a guidewire. In this alternate configuration, the guidewire separator device 100 can optionally be used as a guidewire introducer.

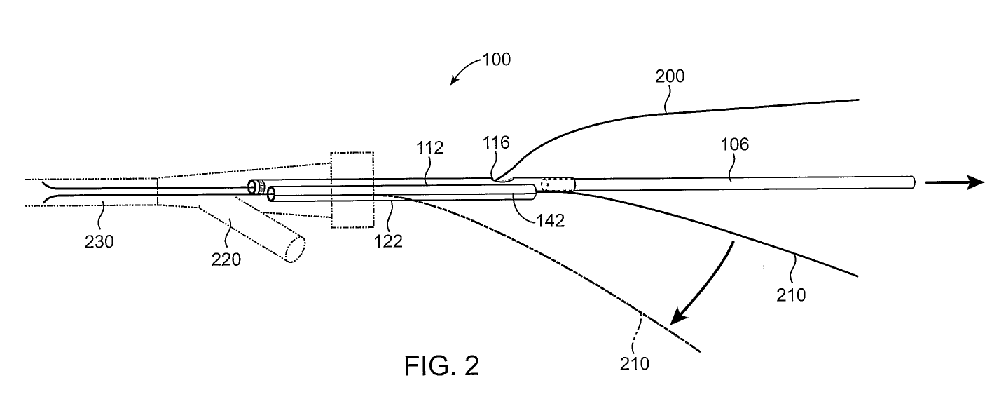

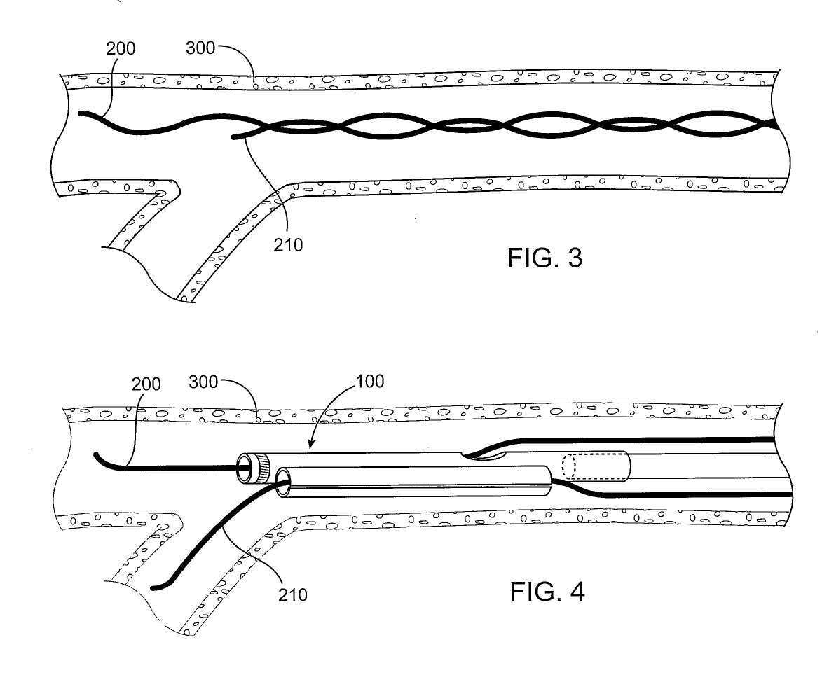

FIG. 3 shows a situation in which an attempt to catheterize two branches of a bifurcated vessel 300 has resulted in twisting and entanglement of the distal ends of the guidewires 200, 210. To remedy this situation, the guidewire separator device 100 is introduced over the proximal ends of the two guidewires 200, 210, as shown by the insertion arrow in FIG. 2, with the first guidewire 200 positioned in the first lumen 110 and the second guidewire 210 positioned in the second lumen 120. The operator holds the proximal ends of the two guidewires 200, 210 and introduces the distal portion 104 of the guidewire separator device 100 into the Y-connector 220 and through the guiding catheter 230 (see FIG. 2). As the distal portion 104 of the guidewire separator device 100 advances along the first and second guidewires 200, 210, it untwists, disentangles and separates the two guidewires 200, 210.

FIG. 4 shows the guidewire separator device 100 within the bifurcated vessel 300 after separating the entangled distal ends of the guidewires 200, 210. Once the guidewires 200, 210 have been separated, the guidewire separator device 100 assists in placement of the guidewires 200, 210 into their respective branches of the bifurcation 300. The guidewire separator device 100 assists in this regard by holding the guidewire closer to the opening of the branch vessel, limiting the lateral movement of the guidewire and providing a fixed axis for precisely rotating and advancing the steerable tip of the guidewire. For this reason, the guidewire separator device 100 may also be used in situations where difficulty in engaging a branch vessel with the guidewire has been encountered or is anticipated based on the vascular anatomy seen on fluoroscopy.

Once the first and second guidewires 200, 210 have been separated and successfully positioned in their respective branches of the bifurcation 300, the guidewire separator device 100 is withdrawn while holding the proximal ends of the guidewires 200, 210 to maintain their positions, as shown by the withdrawal arrow in FIG. 2. The angioplasty or stent deployment catheter or catheter system can then be advanced over the guidewires 200, 210 to complete the procedure as planned.

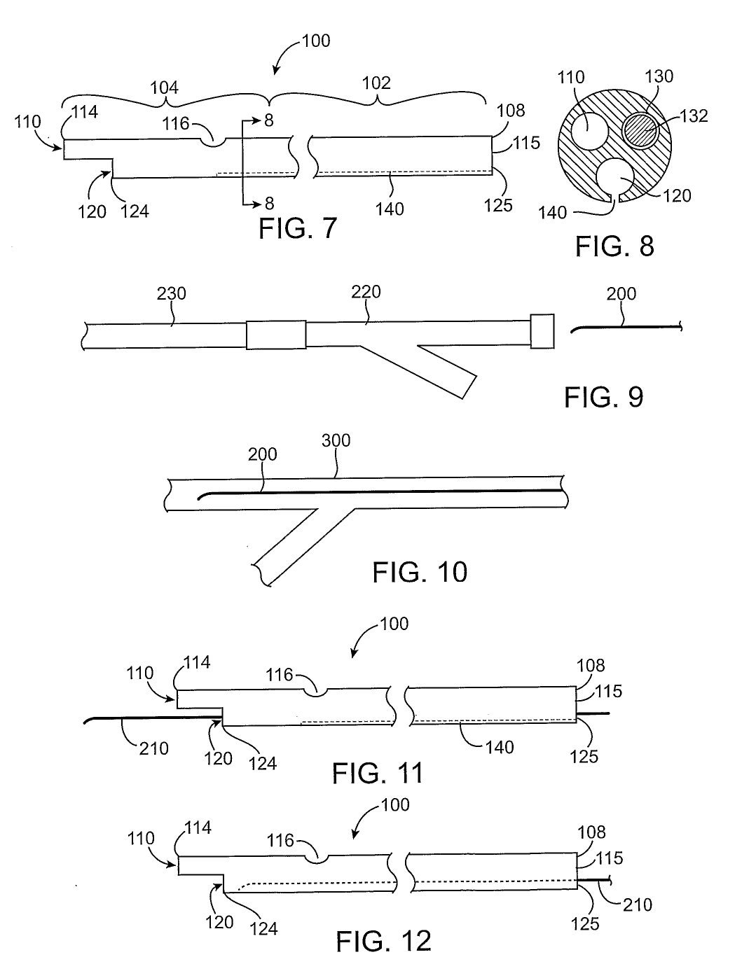

FIG. 7 shows an embodiment of the guidewire separator device 100 that is configured to also function as a guidewire introducer. FIG. 8 shows a cross section of the guidewire separator device 100 of FIG. 7 taken at section line 8-8. In a preferred embodiment for use in coronary artery angioplasty or stenting procedures, the guidewire separator device 100 is divided into a proximal portion 102 with a length of approximately 120 cm and a distal portion 104 with a length of approximately 20 cm. The guidewire separator device 100 has a first guidewire lumen 110 and a second guidewire lumen 120. The distal end of the first guidewire lumen 110 terminates in a first distal guidewire port 114. There is rapid exchange guidewire port 116, which may be a skive or hole formed in the side of the first guidewire lumen 110, at the transition between the proximal portion 102 and the distal portion 104. Optionally, the first guidewire lumen 110 may continue through the proximal portion 102 of the guidewire separator device 100 proximal of the rapid exchange guidewire port 116 to a first proximal guidewire port 115 for introducing a guidewire through this lumen from the proximal end of the guidewire separator device 100. Optionally, there may be a longitudinal slit (not shown) along the proximal portion of the first guidewire lumen 110. The distal end of the second guidewire lumen 120 terminates in a second distal guidewire port 124 and the second guidewire lumen 120 continues through the proximal portion 102 of the guidewire separator device 100 to a second proximal guidewire port 125. Preferably, the first guidewire lumen 110 extends 1-2 cm distally of the second distal guidewire port 124. There is a longitudinal slit 140 along at least the proximal portion of the second guidewire lumen 120. The longitudinal slit 140 serves the function of a rapid exchange guidewire port, while the proximal portion of the second guidewire lumen 120 provides additional support and control of the guidewire through the proximal portion 102 of the device 100. Optionally, the longitudinal slit 140 may also continue along the distal portion 104 of the second guidewire lumen 120. Alternatively, the sidewall of the second guidewire lumen 120 may be made splittable along a longitudinal line.

Optionally, the guidewire separator device 100 includes a third lumen 130 into which a stainless steel stylet 132 or other support member may be permanently or removably inserted. Optionally, the stylet 132 may have tapered distal portion to provide a transition in stiffness between the proximal portion 102 and the distal portion 104 of the guidewire separator device 100.

In a preferred embodiment, the guidewire separator device 100 is constructed from a continuous length of three-lumen extruded tubing that is modified as shown in FIG. 7. Alternatively, the guidewire separator device 100 may be assembled from separate pieces of extruded tubing by welding or adhesively bonding the pieces of tubing together. For example, the proximal portion 102 may be made from a single-lumen or two-lumen extruded tube and the distal portion 104 may be made from a two-lumen or three-lumen extruded tube, depending on the combination of features desired. Suitable materials include, but are not limited to polyethylene, polypropylene, polyolefins, polyamides, polyamide copolymers, PTFE, FEP, and any extrudable medical grade polymer with a suitable combination of strength, flexibility and friction characteristics. Optionally, tubing of different materials or different properties may be joined together to provide a transition of stiffness or other characteristics along the length of the guidewire separator device 100.

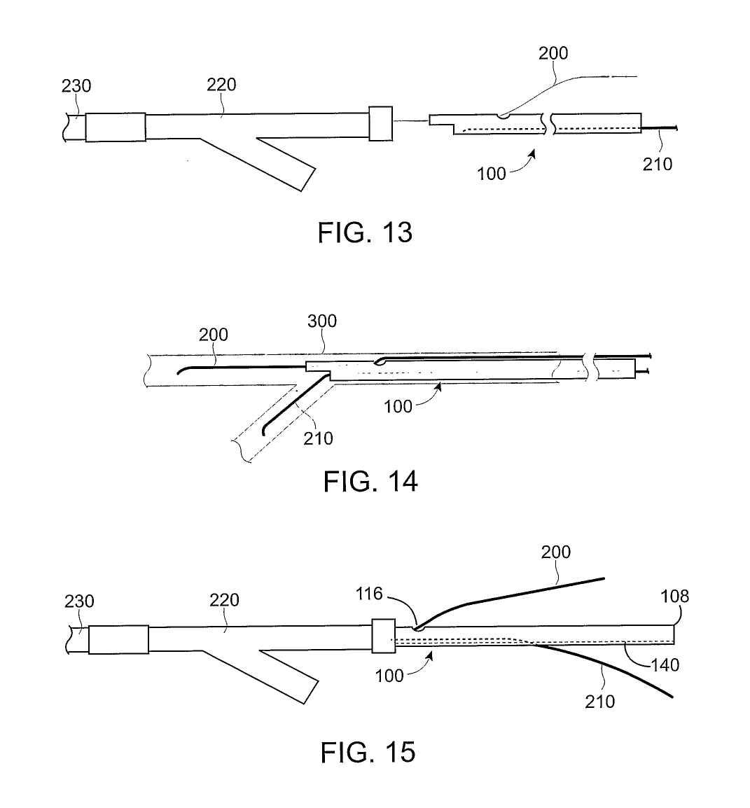

The guidewire separator device 100 can be used to separate two guidewires that have become entangled as described above in connection with FIGS. 3 and 4. In addition, FIGS. 9-15 illustrate a method of using the guidewire separator device 100 of FIG. 7 for introducing a first guidewire 200 and a second guidewire 210 into a bifurcated vessel 300. FIG. 9 shows the first guidewire 200 being introduced through a Y-fitting 220 and a guiding catheter 230, which have been previously placed into the patient's artery. FIG. 10 shows the distal end of the first guidewire 200 being positioned in a first branch (typically the main branch) of the bifurcated vessel 300. The second guidewire 210 is preloaded into the second guidewire lumen 120 of the guidewire separator device 100, as shown in FIGS. 11 and 12. The second guidewire 210 may be loaded into the second guidewire lumen 120 through the second distal guidewire port 124, as shown in FIG. 11, or, alternatively, it may be loaded into the second guidewire lumen 120 through the second proximal guidewire port 125. With the second guidewire 210 preloaded into the second guidewire lumen 120, the proximal end of the first guidewire 200 is threaded into the first distal guidewire port 114, through the distal portion of the first guidewire lumen 110 and out through the first rapid exchange guidewire port 116, as shown in FIG. 13. While holding the proximal end of the first guidewire 200 to prevent it from inadvertently advancing beyond the target location, the guidewire separator device 100 is advanced through the Y-fitting 220 and the guiding catheter 230 until the second distal guidewire port 124 is in the vicinity of the side branch of the bifurcated vessel 300. The guidewire separator device 100 is used to aid in orienting and positioning the second guidewire 210 and the distal end of the second guidewire 210 is advanced into the side branch, as shown in FIG. 14. While holding the proximal ends of the first guidewire 200 and the second guidewire 210, the guidewire separator device 100 is withdrawn from the guiding catheter 230 and through the Y-fitting 220. As the guidewire separator device 100 is withdrawn, the second guidewire 210 is separated from the second guidewire lumen 120 by externalizing it through the slit 140, as shown in FIG. 15. Once the distal portion 104 of the guidewire separator device 100 is completely exposed, the device 100 is removed from the first and second guidewires 200, 210 in the manner of a rapid exchange catheter by grasping them distal to the device 100 and removing the device 100 from the proximal ends of the first and second guidewires 200, 210. The first and second guidewires 200, 210 can now be used to guide first and second rapid exchange angioplasty or stent delivery catheters to the bifurcated vessel 300 to complete the procedure.

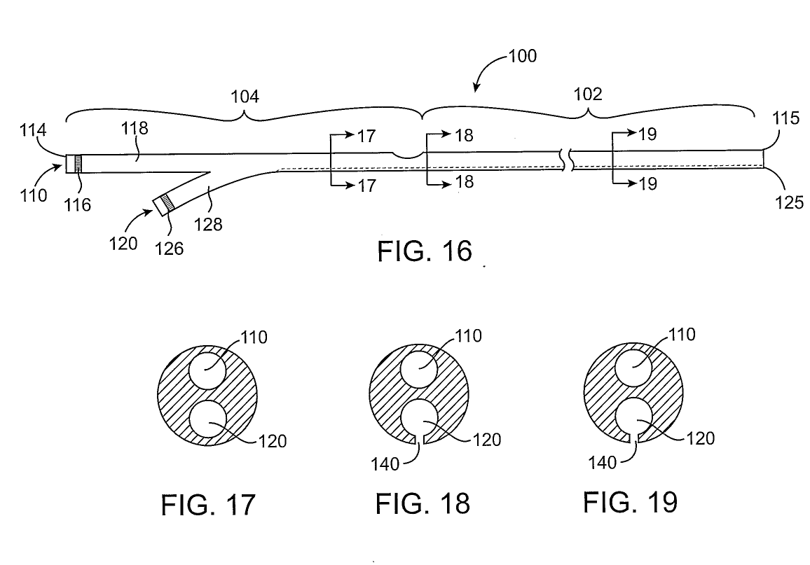

FIG. 16 shows another embodiment of the guidewire separator device 100 configured to function as a guidewire introducer. FIG. 17 shows a cross section of the guidewire separator device 100 of FIG. 16 taken at section line 17-17. FIG. 18 shows a cross section of the guidewire separator device 100 of FIG. 16 taken at section line 18-18. FIG. 19 shows a cross section of the guidewire separator device 100 of FIG. 16 taken at section line 19-19.This embodiment of the guidewire separator device 100 is quite similar to the embodiment described above in connection with FIGS. 7 and 8, except that the distal portion 104 has been modified by separating the first guidewire lumen 110 and the second guidewire lumen 120 for a short distance near the distal end of the device 100. Preferably, the distal extension 118 of the first guidewire lumen 110 has a length of approximately 10-50 mm and the distal extension 128 of the second guidewire lumen 120 is slightly shorter with a length of approximately 5-30 mm. Preferably, there is a second radiopaque marker 126 located near the distal end of the second distal extension 128. Optionally, the guidewire separator device 100 may include a stylet or other support member, as shown in FIG. 8. However, it has been found that preloading the second guidewire 210 into the second guidewire lumen 120, as shown in FIGS. 11 and 12, provides sufficient support for the device 100 in most cases without any additional support member.

The guidewire separator device 100 of FIG. 16 can be used to separate two guidewires that have become entangled, as described in connection with FIGS. 3 and 4, and/or for introducing two guidewires into a bifurcated vessel, as described in connection with FIGS. 9-15.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

1. A guidewire separator device comprising:
an elongate tubular distal portion joined to an elongate proximal portion,
a first lumen extending through at least the tubular distal portion of the guidewire separator device, the first lumen having a first distal guidewire port located near a distal end of the distal portion and a first proximal guidewire port located near a proximal end of the distal portion, a second lumen extending through at least the tubular distal portion of the guidewire separator device, the second lumen having a second distal guidewire port located near a distal end of the distal portion and a second proximal guidewire port located near a proximal end of the second lumen,
and a longitudinal slit in a sidewall of the guidewire separator device that communicates with the second lumen.

2. The guidewire separator device of claim 1, wherein the proximal portion is configured as a solid stylet joined to the tubular distal portion.

3. The guidewire separator device of claim 1, wherein the proximal portion is configured as a stylet with a tapered distal end inserted into a lumen in the tubular distal portion.

4. The guidewire separator device of claim 1, further comprising a stylet that extends through the proximal portion and the tubular distal portion.

5. The guidewire separator device of claim 1, wherein the longitudinal slit extends the length of the tubular distal portion.

6. The guidewire separator device of claim 1, wherein the proximal portion is configured as a solid stylet joined to the tubular distal portion, and the longitudinal slit extends the length of the tubular distal portion.

7. The guidewire separator device of claim 1, wherein the second distal guidewire port is located at a position on the distal portion that is proximal of the first distal guidewire port.

8. The guidewire separator device of claim 1, wherein the proximal portion comprises a tubular lumen aligned with at least one of the first lumen or the second lumen.

9. The guidewire separator device of claim 1, wherein the second lumen extends through the proximal portion of the guidewire separator device and the longitudinal slit extends the length of the proximal portion.

10. The guidewire separator device of claim 1, wherein the second lumen extends through the proximal portion of the guidewire separator device and the longitudinal slit extends the length of the tubular distal portion and the proximal portion.

11. The guidewire separator device of claim 1, further comprising a radiopaque marker located near a distal end of the distal portion.

12. The guidewire separator device of claim 1, wherein the tubular distal portion includes a first tubular distal extension, with the first lumen extending through the first tubular distal extension, and a second tubular distal extension, with the second lumen extending through the second tubular distal extension, the first tubular distal extension being separate from the second tubular distal extension.

13. The guidewire separator device of claim 1, wherein the tubular distal portion includes a first tubular distal extension, with the first lumen extending through the first tubular distal extension, and a second tubular distal extension, with the second lumen extending through the second tubular distal extension, the first tubular distal extension being separate from the second tubular distal extension, the first tubular distal extension being longer than the second tubular distal extension.

14. The guidewire separator device of claim 13, further comprising a first radiopaque marker located near a distal end of the first tubular distal extension and a second radiopaque marker located near a distal end of the second tubular distal extension.

15. A method for separating a first guidewire and a second guidewire in a vessel of a patient, comprising:
providing a guidewire separator device having an elongate tubular distal portion joined to an elongate proximal portion, a first lumen extending through at least the tubular distal portion of the guidewire separator device, the first lumen having a first distal guidewire port located near a distal end of the distal portion and a first proximal guidewire port located near a proximal end of the distal portion, and a second lumen extending through at least the tubular distal portion of the guidewire separator device, the second lumen having a second distal guidewire port located near a distal end of the distal portion and a second proximal guidewire port located near a proximal end of the second lumen;
inserting a proximal end of the first guidewire into the first lumen of the guidewire separator device;
inserting a proximal end of the second guidewire into the second lumen of the guidewire separator device; and
advancing the guidewire separator device distally along the first guidewire and the second guidewire to separate the first guidewire and the second guidewire.

16. The method of claim 15, further comprising:
withdrawing the guidewire separator device proximally along the first guidewire and the second guidewire and externalizing the second guidewire from the second lumen through a longitudinal slit in a sidewall of the guidewire separator device that communicates with the second lumen.

17. A method for introducing a first guidewire and a second guidewire into a bifurcated vessel of a patient, comprising:
providing a guidewire separator device having an elongate tubular distal portion joined to an elongate proximal portion, a first lumen extending through at least the tubular distal portion of the guidewire separator device, the first lumen having a first distal guidewire port located near a distal end of the distal portion and a first proximal guidewire port located near a proximal end of the distal portion, and a second lumen extending through at least the tubular distal portion of the guidewire separator device, the second lumen having a second distal guidewire port located near a distal end of the distal portion and a second proximal guidewire port located near a proximal end of the second lumen;
loading second guidewire into the second lumen of the guidewire separator device;
inserting the first guidewire into a first branch of the bifurcated vessel;
inserting a proximal end of the first guidewire into the first lumen of the guidewire separator device;
advancing the guidewire separator device into the bifurcated vessel along the first guidewire;
and advancing the second guidewire though the second lumen of the guidewire separator device and into a second branch of the bifurcated vessel.

18. The method of claim 17, further comprising:
withdrawing the guidewire separator device proximally along the first guidewire and the second guidewire and externalizing the second guidewire from the second lumen through a longitudinal slit in a sidewall of the guidewire separator device that communicates with the second lumen.

19. A guidewire separator device comprising:
an elongate shaft having a proximal end and a distal end,
a first guidewire lumen extending through the elongate shaft from a first distal guidewire port located at the distal end of the elongate shaft to a first rapid exchange guidewire port located at a position between the proximal end and the distal end of the elongate shaft,
a second guidewire lumen extending through the elongate shaft from a second distal guidewire port located at a position between the distal end of the elongate shaft and the first rapid exchange guidewire port to a second proximal guidewire port located at the proximal end of the elongate shaft.

20. The guidewire separator device of claim 19, further comprising a first radiopaque marker located on the elongate shaft near the first distal guidewire port.

21. The guidewire separator device of claim 20, further comprising a second radiopaque marker located on the elongate shaft near the second distal guidewire port.

22. The guidewire separator device of claim 19, wherein the elongate shaft is approximately 115-180 centimeters long.

23. The guidewire separator device of claim 19, wherein the second distal guidewire port is located approximately 1-2 centimeters proximal to the distal end of the elongate shaft.

24. The guidewire separator device of claim 19, wherein the first rapid exchange guidewire port is located approximately 15-20 centimeters proximal to the distal end of the elongate shaft.

25. The guidewire separator device of claim 19, further comprising a proximal extension of the first guidewire lumen extending through the elongate shaft from the first rapid exchange guidewire port to the proximal end of the elongate shaft, and a first longitudinal slit in a side wall of the elongate shaft that communicates with the proximal extension of the first guidewire lumen.

26. The guidewire separator device of claim 19, further comprising a second longitudinal slit in a side wall of the elongate shaft that communicates with the second guidewire lumen.

27. The guidewire separator device of claim 19, further comprising a metal stylet that extends through at least a portion of the elongate shaft.

28. The guidewire separator device of claim 19, further comprising:
a first radiopaque marker located on the elongate shaft near the first distal guidewire port,
a second radiopaque marker located on the elongate shaft near the second distal guidewire port,
wherein the elongate shaft is approximately 115-180 centimeters long, the second distal guidewire port is located approximately 1-2 centimeters proximal to the distal end of the elongate shaft, and the first rapid exchange guidewire port is located approximately 15-20 centimeters proximal to the distal end of the elongate shaft.
